(19) United States
(12) Patent Application Publication
(10) Pub. No.: US 2025/0384982 A1
(43) Pub. Date: Dec. 18, 2025

(54) Deduplicating And Grouping Medication Events Using Concept Mapping Of Free Text With Large Language Models

(71) Applicant: Cerner Innovation, Inc., Kansas City, MO (US)

(72) Inventors: Suman Pal, Bangalore (IN); Chetan KV, Bangalore (IN); Monica Gaur, Delhi (IN); Rupanjali Chaudhuri, Bangalore (IN); Margaret Mary Jackson, Collegeville, PA (US); Sourav Gantait, Kolkata (IN); Michael Chung Kun Chen, San Jose, CA (US)

(73) Assignee: Cerner Innovation, Inc., Kansas City, MO (US)

(21) Appl. No.: 18/742,412

(22) Filed: Jun. 13, 2024

(51) Int. Cl. G16H 20/10; G16H 50/20

(52) U.S. Cl. CPC G16H 20/10; G16H 50/20

(57) ABSTRACT

Techniques for generating recommendations of standard medication codes for storing in association with medication free text to facilitate deduplication of patient medication events are disclosed. Standard medication codes are alphanumeric identifiers that represent medication events. Medication free text is medication event information in natural language. The system generates vector embeddings for the standard medication codes by applying a vector embedding function to a set of attributes associated with the standard medication codes. The system generates a vector embedding for a target unmapped medication code by applying the vector embedding function to medication free text of the target unmapped medication code. The system compares the target vector embedding for the target unmapped medication code to the vector embeddings computed for each of the standard medication codes. The system presents recommended standard medication codes and groupings of similar standard medication codes to a user for mapping to the medication free text.

### TECHNICAL FIELD

The present disclosure relates to data deduplication of medication event records. In particular, the present disclosure relates to deduplicating medication events associated with free text using natural language processing.

### BACKGROUND

In fostering an open and collaborative healthcare landscape for effective communication among diverse electronic health record (EHR) platforms, progress has been made over the past decade in enabling health data exchange. This has resulted in an abundance of information, particularly as relates to a patient's healthcare history, where the patient may have seen multiple healthcare providers belonging to different organizations or healthcare systems. The abundance of information may include redundant or duplicate information.

Prior to prescribing or otherwise administering a medication to a patient, healthcare providers consult with the patient's medication history. Patient medication data includes medication events identified with alphanumeric medication codes, e.g., standard medication codes or propriety medication codes associated, or identified with medication free text. Patient medication data may be retrieved from numerous sources. Data for individual patient medication events may be received from multiple separate sources, and each source may identify the same patient medication event in a different manner, e.g., standard medication code, proprietary medication code, and medication free text, resulting in duplication of patient medication data.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIGS. 2A-2C illustrates an example set of operations for deduplicating medication events using natural language processing in accordance with one or more embodiments;

FIGS. 3A-3C illustrate an example system for deduplication medication events; and

FIG. 4 illustrates an example of data flow during an example set of operations for presenting a recommendation of candidate standard medication codes for medication free text;

FIG. 5 illustrates an interface for presenting recommendations of candidate standardized medication codes for medication free text; and

FIG. 6 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.

### 1. GENERAL OVERVIEW

### 2. MEDICATION SYNCHRONIZATION SYSTEM

### 3. PRESENTING RECOMMENDATIONS OF CANDIDATE STANDARD MEDICATION CODES FOR MAPPING TO UNMAPPED MEDICATION CODES TO FACILITATE MEDICATION EVENT DEDUPLICATION

### 4. EXAMPLE SYNCHRONIZATION SYSTEM

### 5. EXAMPLE MAPPING OPERATIONS

### 6. RECOMMENDATION INTERFACE

### 7. PRACTICAL APPLICATIONS, ADVANTAGES & IMPROVEMENTS

### 8. HARDWARE OVERVIEW

### 9. MISCELLANEOUS; EXTENSIONS

### 1. General Overview

One or more embodiments generate recommendations of standard medication codes for storing in association with medication free text to facilitate deduplication of patient medication events. Standard medication codes, as referred to herein, are alphanumeric identifiers that represent medication events. Standard medication code sets are developed and maintained by organizations and industries involved in healthcare information management, regulation, and standardization. Standard medication codes facilitate the electronic exchange of medication-related information between different healthcare systems and organizations. Medication free text, as referred to herein, is medication event information in natural language.

Initially, the system generates vector embeddings for the standard medication codes by applying a vector embedding function to a set of attributes associated with the standard medication codes. Applying a vector embedding function to the set of attributes includes applying the vector embedding function to text of the set of attributes.

In one or more embodiments, a target unmapped medication code is represented by medication free text. The system generates a vector embedding for the target unmapped medication code by applying the vector embedding function to the medication free text of the target unmapped medication code. The system may generate a vector embedding for the medication free text at least by applying the vector embedding function to an aggregate of the text of the medication free text. Alternatively, or in addition, the system may apply the vector embedding function to each instance of the medication free text and combine the resulting vector embeddings to generate the vector embedding for the unmapped medication code. The text associated with each unmapped medication code may be pre-processed or otherwise normalized prior to application of the vector embedding function. Pre-processing or normalizing may include, for example, filtering out certain words, handling special characters, and replacing abbreviations with full form text.

In an embodiment, the system compares the target vector embedding for the target unmapped medication code to the vector embeddings computed for each of the standard medication codes. Based on a similarity measure between the target vector embedding and the vector embeddings for the unmapped medication code, the system selects a subset of the standard medication codes for recommending to the user as a set of candidate standard medication codes for the target unmapped medication code. The system presents a group of standard medication codes, including the standard medication code and similar medication codes to a user for selection. Upon receipt of user input selecting a particular standard medication code, of the set of candidate standard medication codes, the system stores an association, or mapping, between the medication free text and the particular standard medication code.

In one or more embodiments, the system identifies a second medication event associated with a second standard medication code as being the same as the first standard medication code. The system removes one of the first or second medication event from the patient medication data as being duplicative of the other of the first or second medication event.

One or more embodiments described in this Specification and/or recited in the claims may not be included in this General Overview section.

### 2. Medication Synchronization System

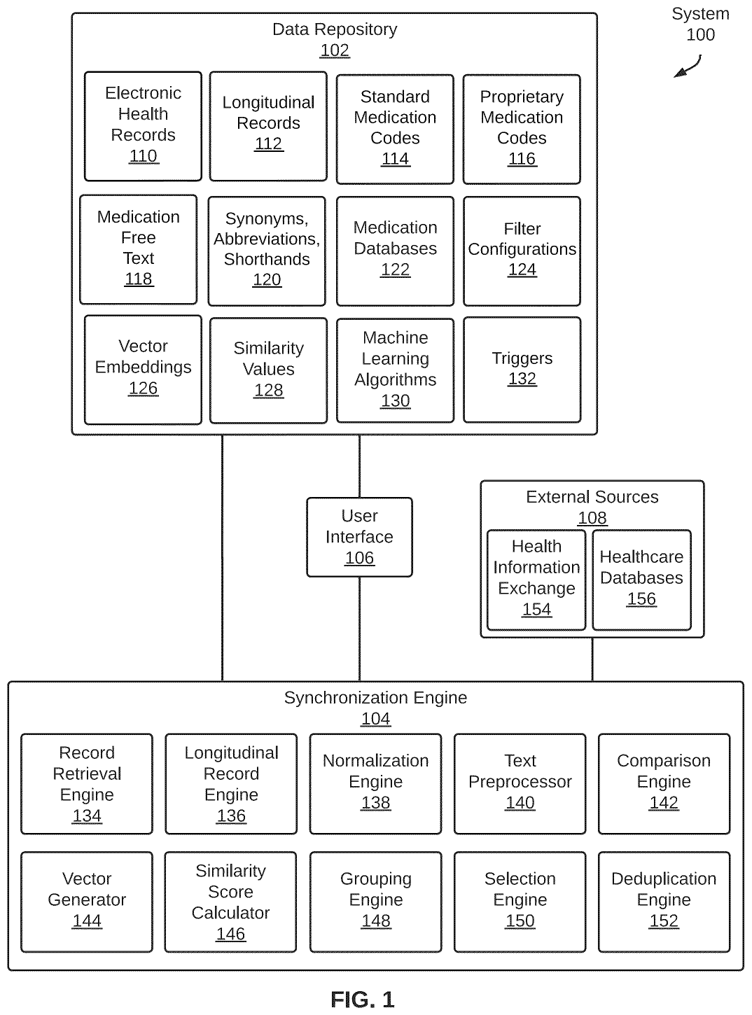

FIG. 1 illustrates a system 100 in accordance with one or more embodiments. As illustrated in FIG. 1, system 100 includes a data repository 102, a synchronization engine 104, a user interface 106, and external sources 108. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Further, a data repository 102 may include multiple different storage units and/or devices. The multiple different storage units and/or devices may or may not be of the same type or located at the same physical site. Further, a data repository 102 may be implemented or executed on the same computing system as the synchronization engine 104 and the user interface 106. Additionally, or alternatively, a data repository 102 may be implemented or executed on a computing system separate from the synchronization engine 104 and the user interface 106. The data repository 102 may be communicatively coupled to the synchronization engine 104 and the user interface 106 via a direct connection or via a network.

Information describing operations for deduplicating patient medication events using nature language processing may be implemented across any of components within the system 100. However, this information is illustrated within the data repository 102 for purposes of clarity and explanation.

In embodiments, the data repository 102 is populated with information from a variety of sources and/or systems. The data repository 102 may include electronic healthcare records (EHRs) 110, longitudinal records 112, standard medication codes 114, proprietary medication codes 116, medication free text 118, synonyms, abbreviations, and shorthands 120, a medical database 122, filter configurations 124, vector embeddings 126, similarity values 128, machine learning algorithms 130, and triggers 132. Any of this information may be stored in a structured format (e.g., a table).

In one or more embodiments, EHRs 110 are digital versions of healthcare records. EHRs 110 comprise medical history, diagnoses, medications, treatment plans, immunization dates, allergies, radiology images, laboratory test results, and/or other patient information. EHRs 110 may be from the same or different systems and/or providers. Some examples of EHR providers include Cerner Millenium and Epic.

In embodiments, EHRs 110 are populated with medication codes associated with patient medication events. Patient medication events include instances of a patient being prescribed a medication—whether taken by the patient or not, medications taken by the patient, e.g., over-the-counter, and/or medication administered to the patient. The medication codes associated with the patient medication events may include standard medication codes 114, proprietary medication codes 116, medication free text 118, and/or a combination of these. For example, a first EHR may identify patient medication events with proprietary medication codes and a second EHR may identify patient medication events with medication free text.

In one or more embodiments, longitudinal records 112 are comprehensive and cumulative records that document a patient's health information over time. Unlike traditional medical records, which may only capture a snapshot of a patient's health status at a specific point in time, longitudinal records provide a longitudinal view of the patient's health history, diagnoses, treatments, medications, procedures, and outcomes across multiple encounters and care settings.

In embodiments, longitudinal records 112 offer continuity, comprehensiveness, timeliness, accessibility, and interoperability. Longitudinal records 112 span the entire continuum of care, capturing information from various healthcare encounters, including primary care visits, specialist consultations, hospitalizations, emergency department visits, diagnostic tests, and procedures. This continuity of information provides healthcare providers with a comprehensive understanding of the patient's health trajectory and medical history. Longitudinal records 112 encompass a wide range of health information, including medical history, social history, family history, allergies, medications, immunizations, laboratory results, imaging studies, progress notes, care plans, and outcomes. This comprehensive view enables healthcare providers to make informed decisions about diagnosis, treatment, and care management.

In some embodiments, longitudinal records 112 are updated in real-time or near-real-time as new health information becomes available. This timely updating ensures that healthcare providers have access to the most current and accurate patient information when making clinical decisions or providing care. Longitudinal records 112 are accessible to authorized healthcare providers and patients across different care settings and healthcare organizations. EHR systems, health information exchanges (HIEs), and patient portals facilitate the sharing and exchange of longitudinal health information while maintaining patient privacy and security. Longitudinal records 112 support interoperability between different healthcare systems and applications, allowing seamless exchange and integration of health information across disparate platforms. Standards-based data exchange protocols, terminologies, and coding systems promote interoperability and data exchange among healthcare stakeholders.

In one or more embodiments, standard medication codes 114 are alphanumeric identifiers used to represent medications in healthcare settings. Standard medication code sets are developed and maintained by organizations and industries involved in healthcare information management, regulation, and standardization. Standard medication codes 114 facilitate the electronic exchange of medication-related information between different healthcare systems and organizations.

Some widely used standard medication code systems include Systematized Nomenclature of Medicine Clinical Terms (SNOMED CT), Anatomical Therapeutic Chemical Classification System (ATC), LOINC (Logical Observation Identifiers Names and Codes), and Health Level Seven (HL7) Standard Codes. The NDC is a unique 10-digit, 3-segment numeric identifier assigned to medications in the United States by the Food and Drug Administration (FDA). The NDC identifies the manufacturer or distributor, the product, and the package size or dosage form of the medication. SNOMED CT is a comprehensive clinical terminology system used internationally to represent clinical concepts in healthcare. SNOMED CT includes codes for medications, as well as other clinical concepts, procedures, and observations, to support interoperability and semantic interoperability in healthcare information systems. The ATC system is an international classification system developed by the World Health Organization (WHO) for the classification of drugs based on their therapeutic and pharmacological properties. The ATC system uses alphanumeric codes to categorize medications into different anatomical groups, therapeutic groups, and chemical subgroups. While primarily used for laboratory tests and clinical observations, LOINC also includes codes for clinical drug names and medication-related concepts to support interoperability in electronic health records (EHRs) and health information exchanges (HIEs). HL7includes standard code systems for representing medications, such as those used in HL7 Version 2 and HL7 Version 3 messaging standards, to facilitate the exchange of medication-related information between healthcare systems and applications.

In one or more embodiments, the standard medication codes 114 are RxNorm. RxNorm is a standardized nomenclature for clinical drugs developed by the National Library of Medicine (NLM). RxNorm provides normalized names and codes for clinical drugs, including brand names, generic names, and ingredients, to facilitate electronic prescribing and medication reconciliation. RxNorm uses Term Types (TTYs) to indicate generic and branded drug names at different levels of specificity. TTYs are semantic tags that describe the type of information the concept conveys.

TTYs include route, dosage, ingredient (IN), precise ingredient (PIN), multiple ingredients (MIN), semantic clinical drug (SCD), semantic branded drug (SBD), brand name pack (BPCK), and generic pack (GPCK). Route refers to the path or method by which a medication is administered or delivered into the body. Route specifies how a drug is introduced to the patient's system, indicating whether the drug is taken orally, injected, applied topically, inhaled, or administered through other routes. Common examples of medication routes include oral (by mouth), intravenous (IV), intramuscular (IM), subcutaneous (SC), topical (applied to the skin), and inhalation. Dosage refers to the specific amount or quantity of a drug prescribed for an individual patient during a given period. It is a crucial component of a medical prescription and is often expressed in terms of units of the drug (such as milligrams or micrograms) per unit of the patient (such as kilograms or pounds) and the frequency of administration (such as once daily or twice a day). Ingredient (IN) is a compound or moiety that gives the drug its distinctive clinical properties. Ingredients generally use the United States Adopted Name (USAN). Example: Fluoxetine. Precise Ingredient (PIN) is a specified form of the ingredient that may or may not be clinically active. The most precise ingredients are salt or isomer forms. Example: Fluoxetine Hydrochloride Multiple Ingredients (MIN) are two or more ingredients appearing together in a single drug preparation, created from Semantic Clinical Drug Form (SCDF). Clinical Drug (SCD): Ingredient+Strength+Dose Form. Example: Fluoxetine 4 MG/ML Oral Solution. Semantic Branded Drug (SBD): Ingredient+Strength+Dose Form+Brand Name. Example: Fluoxetine 4 MG/ML Oral Solution [Prozac]. Brand Name Pack (BPCK) is {# (Ingredient Strength Dose Form)/# (Ingredient Strength Dose Form)} Pack [Brand Name]. Example: {12 (Ethinyl Estradiol 0.035 MG/Norethindrone 0.5 MG Oral Tablet)/9 (Ethinyl Estradiol 0.035 MG/Norethindrone 1 MG Oral Tablet)/7 (Inert Ingredients 1 MG Oral Tablet)} Pack [Leena 28 Day]. Generic Pack (GPCK) is {# (Ingredient+Strength+Dose Form)/# (Ingredient+Strength+Dose Form)} Pack. Example: {11 (varenicline 0.5 MG Oral Tablet)/42 (varenicline 1 MG Oral Tablet)} Pack.

In one or more embodiments, proprietary medication codes 116 are identifiers specific to particular healthcare organizations, pharmacy chain, or electronic health record (EHR) system. Unlike standard medication codes 114, which follow universally accepted standards and are designed for interoperability between different systems, proprietary medication codes 116 are internal to a specific organization's database or system. The proprietary medication codes 116 may be used for various purposes within the organization, including inventory management, billing, internal communication, and data analytics. Proprietary medication codes 116 often provide additional information or functionality tailored to the organization's specific needs and workflows.

An example of a proprietary medication code system is the “GEM” codes used by the Veterans Health Information Systems and Technology Architecture (VistA) electronic health record system, which is widely used within the United States Department of Veterans Affairs (VA) healthcare system. GEM stands for “Generic Equivalent Medication” codes, and they are internal identifiers used within VistA to represent medications and drug products. These codes are specific to the VA's medication catalog and are not part of any universally accepted standard code system. Each medication in the VA's formulary is assigned a unique GEM code, which is used for various purposes within the VistA system, including prescribing, dispensing, inventory management, and billing. For example, a proprietary medication code in the VistA system might look like: GEM12345: Acetaminophen 500 mg Tablet. In this example, “GEM12345” would be the proprietary medication code used internally by the VA's VistA system to represent the specific formulation of acetaminophen tablets.

In one or more embodiments, medication free text 118 refers to patient medication events identified using natural language or plain text. In this manner, healthcare providers document medication events using plain text, rather than selecting medications from a predefined list or database. In healthcare settings, health care providers may have the option to enter medication orders or prescriptions using free text fields in electronic health record (EHR) systems or prescribing software. Medication free-text may include medication names that do not match standard drug names or codes, abbreviations, acronyms, or shorthand notations that are not standard or universally recognized, descriptions of medication regimens, dosing instructions, or administration schedules that are not in a structured format, and notes, comments, or annotations associated with medication events that provide additional context or information but are not coded or standardized.

In embodiments, medication free text 118 provides flexibility and allows healthcare providers to document medications in a format preferred by the healthcare provider. The use of medication free text 118 introduces challenges related to accuracy, standardization, and interoperability. Medication free text is prone to errors, such as misspellings, abbreviations, or incomplete information. These errors can lead to misinterpretation by other healthcare providers.

In some embodiments, the synonyms, abbreviations, and shorthands 120 are included in a table that provides synonyms, abbreviations, and/or shorthands that may or may not be specific to a consumer and corresponding expansions for the respective synonym, abbreviation or shorthand. For example, “qd” may refer to once a day, “bid” may refer to twice a day, “ac” may refer to before meals, “po” may refer to orally, “q4h” may refer to every four hours, and “qod” may refer to every other da.

In one or more embodiments, medication database 122 is a structured collection of data containing comprehensive information about medications, e.g., Multum, Lexicomp, Micromedex. The medication database 122 serves as a central repository of medication-related data that can be accessed, queried, and utilized by healthcare professionals, researchers, and software applications for various purposes, such as prescribing, dispensing, administration, monitoring, and research. The medication database 122 may include the standard medication codes 114, proprietary medication codes 116, medication free text 118, synonyms, abbreviations, shorthands 120, code mappings, and medication code groupings.

In one or more embodiments, medication databases 122 include medication information, drug interactions, and clinical guidelines. Medication information is detailed information about medications, including generic and brand names, dosages and strengths, routes of administration, formulations and dosage forms, indications and uses, contraindications and warnings, and side effects and adverse reactions. Drug Interactions are information about potential interactions between medications, including drug-drug interactions, drug-food interactions, drug-allergy interactions, pharmacokinetic interactions, pharmacodynamic interactions. Clinical Guidelines are recommendations and guidelines for safe and effective medication use, including dosage recommendations, administration guidelines, monitoring parameters, special populations considerations (e.g., pediatric, geriatric, pregnancy), and treatment algorithms and protocols.

In one or more embodiments, medication databases 122 includes formulary management, coding and classification, and references and citations. Formulary management is information about medications included in healthcare organization formularies, including preferred drug lists, drug utilization reviews, therapeutic interchange programs, medication cost and reimbursement information. Regulatory information is compliance and regulatory data related to medications, including: FDA approvals and labeling information, drug scheduling and controlled substance classifications, black box warnings and safety alerts, post-marketing surveillance data. Coding and classification is standardized coding systems and classification schemes for medications, such as NDC, RxNorm, ATC, SNOMED CT. This may include mappings for mapping a first set of standard medication codes to a second set of standard medications codes, or mapping standard medication codes 114 to proprietary medication codes 116, and/or mapping standard medication codes 114 to medication free text 118. Mappings may also include mappings between inactive codes and active codes. Coding and classification may also include groupings of like or similar standard medication codes 114, e.g., brand name and generic medications. References and citations are sources of medication information, including pharmacology textbooks and reference books, clinical practice guidelines, research articles and scientific literature, drug manufacturer package inserts.

In one or more embodiments, the filter configurations 124 determine how a normalization engine 138 of the synchronization engine 104 filters and sorts the patient medication events. The patient medication data may be sorted by medication code into like or similar groups or buckets. Patient medication events identified with standard medication codes may be separated from patient medication events identified with medication free text. Patient medication events not associated with a known medication code or medication free text may be removed from the patient medication data.

In one or more embodiments, the vector embeddings 126 in the data repository 102 include text that has been converted to a numeric format. The vector embeddings 126 are representations of individual words for text analysis, typically in the form of a real-valued vector. The vector embeddings 126 may represent individual text items or may represent an aggregation of text items. As will be described in further detail below with respect to synchronization engine 104, the vector embeddings 126 may be formed using various word embedding techniques. The vector embeddings 126 represent standard medication codes and medication free text.

In some embodiments, the similarity values or metrics 128 in the data repository 102 provide an indication of the similarity between the vector embeddings 126 for standard medication codes and medication free text. The higher the similarity values 128 (for example, the closer to 1.0, depending on the scale), the greater a semantic match between the vector embeddings 126 of a standard medication code and a medication free text. The similarity values 128 may be assigned a ranking category. For example, a similarity value less than 0.90 may be categorized as “low”; a similarity value equal to or greater than 0.90 and less than 0.98 may be categorized as “medium”; and a similarity value greater than or equal to 0.98 may be categorized as “high.” The similarity values 112 may be weighted to reflect the relevance of the type of data used to calculate the vector embeddings 126.

In one or more embodiments, a machine learning algorithm 130 is an algorithm that can be iterated to train a target model f that best maps a set of input variables to an output variable. In particular, a machine learning algorithm 130 is configured to generate and/or train a semantic similarity model or a deduplication model.

A machine learning algorithm is an algorithm that can be iterated to train a target model f that best maps a set of input variables to an output variable, using a set of training data. The training data includes datasets and associated labels. The datasets are associated with input variables for the target model f. The associated labels are associated with the output variable of the target model f. The training data may be updated based on, for example, feedback on the predictions by the target model f and accuracy of the current target model f. Updated training data is fed back into the machine learning algorithm, which in turn updates the target model f.

A machine learning algorithm 130 generates a target model f such that the target model f best fits the datasets of training data to the labels of the training data. Additionally, or alternatively, a machine learning algorithm 130 generates a target model f such that when the target model f is applied to the datasets of the training data, a maximum number of results determined by the target model f matches the labels of the training data. Different target models may be generated based on different machine learning algorithms and/or different sets of training data.

A machine learning algorithm may include supervised components and/or unsupervised components. Various types of algorithms may be used, such as linear regression, logistic regression, linear discriminant analysis, classification and regression trees, naïve Bayes, k-nearest neighbors, learning vector quantization, support vector machine, bagging and random forest, boosting, backpropagation, and/or clustering.

In one or more embodiments, triggers 132 for automatic synchronization of medical events or conditions that initiate the synchronization process between different systems or devices without requiring manual intervention. Triggers 132 automate the synchronization workflow, ensuring that data remains consistent and up to date across all synchronized endpoints.

In one or more embodiments, triggers 132 include scheduled synchronization, event-based triggers, threshold-based triggers, system startup or shutdown, manual override, dependency-based triggers, external events or conditions. Synchronization can be scheduled to occur at specific intervals, such as hourly, daily, or weekly. Synchronization can be triggered by specific events or actions, such as the creation, modification, or deletion of data records, scheduling of future appointments, admittance, discharge and transfer of a patient. For example, when a new record is added to one system, an event-based trigger can initiate synchronization to propagate the new record to other synchronized systems in real-time. Synchronization can be triggered based on predefined thresholds or conditions. For example, synchronization may be triggered when the number of pending changes exceeds a certain threshold or when a specific data condition is met. Synchronization can be triggered automatically when a system or application starts up or shuts down.

While automatic triggers handle most synchronization scenarios, manual triggers can also be implemented to allow users to initiate synchronization manually when needed. Manual override triggers provide flexibility for users to synchronize data on-demand, especially in situations where immediate synchronization is required. Synchronization can be triggered based on dependencies between data elements or systems. For example, if changes to a particular data element depend on changes to another related data element, synchronization can be triggered automatically when the dependent data element is modified. Synchronization can be triggered by external events or conditions detected by external systems or sensors. For example, synchronization may be triggered in response to changes in environmental conditions, healthcare trends, or other external factors that affect the data being synchronized.

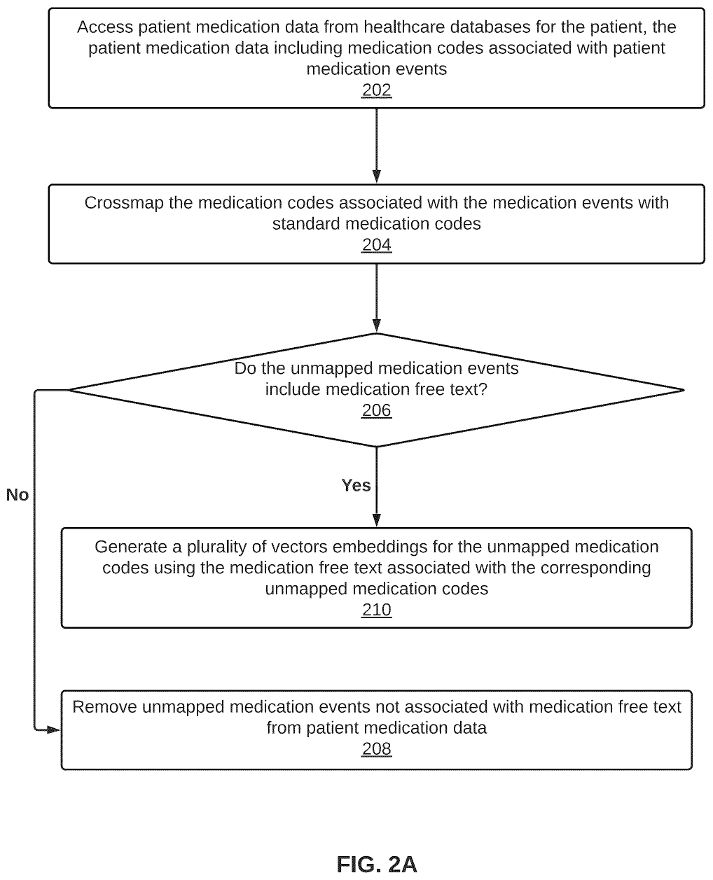

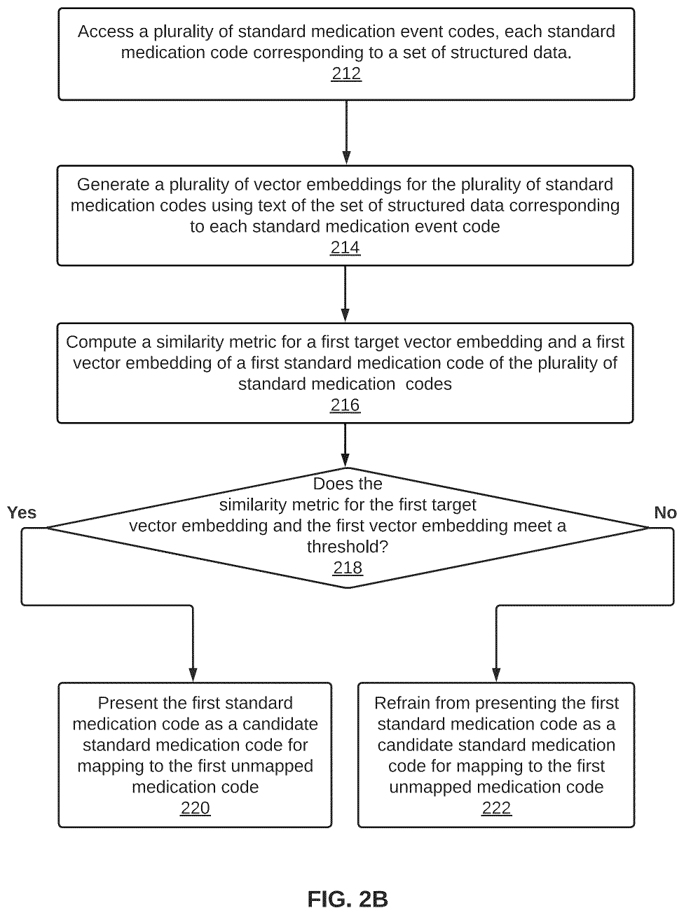

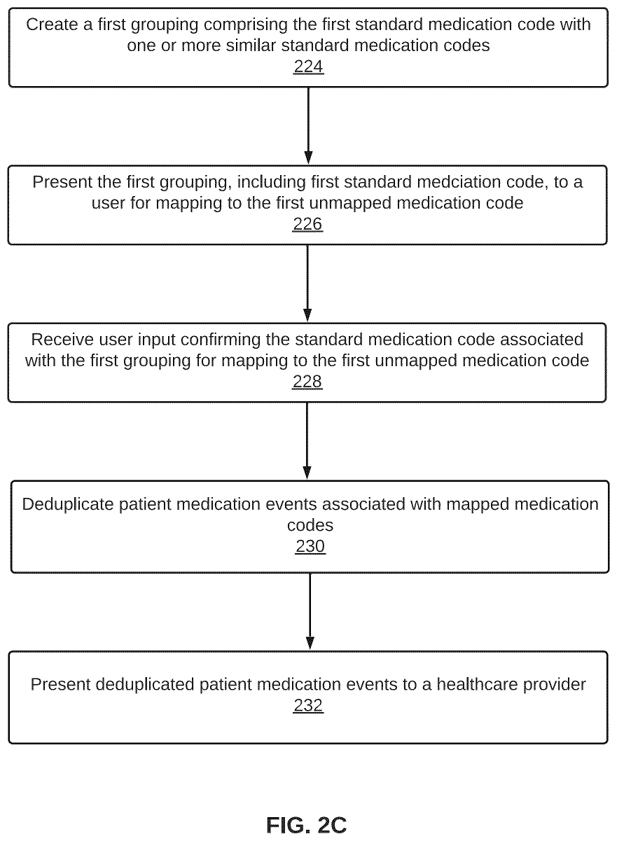

In one or more embodiments, synchronization engine 104 refers to hardware and/or software configured to perform operations described herein for deduplicating patient medication records using natural language processing to map medication free text to standard medication codes. Examples of operations for mapping medication free text to standard medication codes to assist in deduplicating patient medication records are described below with reference to FIGS. 2A-2C.

In an embodiment, synchronization engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, the synchronization engine 104 includes a record retrieval engine 134, a longitudinal record engine 136, a normalization engine 138, a text preprocessor 140, a comparison engine 142, a vector generator 144, a similarity score calculator 146, a grouping engine 148, a selection engine 150, and a deduplication engine 152.

In one or more embodiments, the record retrieval engine 134 is a software component or system that facilitates the retrieval of records or data from one or more databases or data repositories based on specified criteria or queries. The record retrieval engine 134 processes user queries or search criteria to identify relevant records or data within the database. Users can specify criteria such as keywords, filters, or conditions to narrow down the search and retrieve specific records. The record retrieval engine 134 aggregates data from various sources and healthcare encounters, including EHRs, hospital information systems (HIS), laboratory information systems (LIS), radiology information systems (RIS), pharmacy systems, and other clinical data repositories. Alternatively, data retrieval and aggregation are provided by the record retrieval engine 134. The record retrieval engine 134 consolidates disparate data sources to create a unified and comprehensive view of the patient's health information. The record retrieval engine 134 integrates with different healthcare systems and applications using interoperability standards and interfaces, such as HL7, FHIR, or proprietary Application Programming Interfaces (APIs). This allows the record retrieval engine 134 to retrieve and harmonize data from multiple sources, regardless of vendor or system type.

In one or more embodiments, the longitudinal record engine 136, also known as a longitudinal health record system or longitudinal patient record system, is a software platform or component designed to organize and present comprehensive health information about an individual patient over time. The longitudinal record engine 136 provides a longitudinal view of the patient's health history, medical encounters, treatments, medications, test results, and other relevant clinical data across different healthcare settings and encounters.

In one or more embodiments, the normalization engine 138 is a software component or system that standardizes and normalizes data from heterogeneous sources, ensuring consistency in terminology, coding, and formatting. The normalization engine 138 maps and translates data elements to standardized vocabularies, coding systems (e.g., SNOMED CT, LOINC, RxNorm), and data models to promote interoperability and semantic consistency. The normalization engine 138 may also perform filtering the patient medication data events based on the filter configurations 124.

In some embodiments, the text preprocessor 140 is a software component or system that performs functions such as converting the text into lowercase, removing white spaces, prefix removal, punctuation removal, and/or retaining numeric tokens. Text is converted to lowercase to provide uniformity to the text. Prefix removal includes removing prefixes such as “z,” “zz,” “zzz.” Punctuation removal is performed to remove any non-alphanumeric characters. In prior art mapping engines, numeric tokens are typically removed during text preprocessing. Removal of numeric tokens may eliminate a distinguishing feature of a medication free text. For example, “lamictal 25 mg tablet” and “lamictal 50 mg tablet” are differentiated using a numeric token. By retaining numeric tokens, misclassifications are more readily avoided.

In embodiments, text preprocessing may further include handling special characters, removing unwanted text, and custom preprocessing. Handling special characters includes addressing symbols and special characters. For example, text line “D-Dimer” requires special attention. Replacing the “-” with a blank space creates two different tokens, namely “D” and “Dimer.” As such, using traditional text preprocessing, the entire context of “D-Dimer” is lost. By addressing special characters, the context of the terms is maintained. Custom preprocessing includes attending to consumer specific text such as synonyms, abbreviations, and shorthands. The custom preprocessing may consult the synonyms, abbreviations, and shorthands 120 stored in the data repository 102 to provide expansions for various consumer specific synonyms, abbreviations, and shorthands.

In one or more embodiments, the comparison engine 142 is a software component or system designed to analyze and match medication codes comprising medication free text against previously mapped medication free text. The comparison engine 142 uses lexical search to identify medication free text matching that has already been mapped to a standard medication code 114.

In some embodiments, the vector generator 144 includes software and/or hardware for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be casily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GloVe), BioWordVec FastText, and Bidirectional Encoder Representation (BERT).

Each of these word embedding techniques includes salient features. The TF-IDF model is designed to give more weight to the words that are very specific to certain documents but give less weight to the words that are more general and occur across most documents. The Word2Vec model represents words in the form of dense vectors by capturing syntactic (grammar) and semantic (meaning) relationships. Given a large enough dataset, the Word2vec model provides strong estimates about a meaning of a word based on the frequency of occurrence of the word in the text. The GLOVE model is an unsupervised learning model that can be used to obtain dense word vectors like the Word2Vec model. The GLOVE model first creates a large word-context, co-occurrence matrix consisting of pairs (word, context). Each element in this matrix represents how often a word or a sequence of words occurs within the context and then applies matrix factorization to approximate this matrix. The BioWord Vec fastText model is 200-dimensional word embeddings trained on PubMed and MIMIC-III data and is the extension of the original BioWordVec that provides fastText word embeddings trained using PubMed and MeSH. A subword embedding model used by the Bio Word Vec fastText model better handles out of vocabulary tokens and improves the quality of the word embeddings. BERT uses encoder-only transformer architecture that learns the contextual relations between words (or subwords) in textual data and converts text into embeddings. BERT is trained on an unsupervised task of ‘Mask Language Model (MLM)’ using text corpora from BooksCorpus and English Wikipedia

In one or more embodiments, the word embedding techniques include Clinical-BERT or Self-Alignment Pretraining for Biomedical Entity Representations (SAPBERT). SAPBERT is a specialized Large Language Model (LLM) that is a pre-trained BERT model trained on Medical Entity Linking (MEL) tasks. MEL maps various entities to unified concepts in the medical knowledge graph. Word representation learning faces a significant challenge due to the existence of heterogeneous names. For example, in healthcare, terms like ‘nostril’ and ‘nare’ are used interchangeably but yield considerably different embedding representations when generated by models not specifically trained for MEL. SAPBERT works on self-alignment of biomedical entity representation such that the semantically similar entities belonging to the same concept are brought closer in the embedding space, thus forming compact clusters. SAPBERT leverages UMLS, the largest collection of biomedical concepts and synonyms and collates the synonyms from various controlled vocabularies, e.g., SNOMED CT, MeSH, Gene Ontology, RxNorm, and OMIM. SAPBERT performs better compared to other variants of BERT like Bio-BERT, Clinical-BERT with respect to the MEL challenges. The SAPBERT model can accurately capture fine-grained semantic relationships and heterogeneous naming in the biomedical domain compared to other variants of BERT. The ability of SAPBERT to handle out-of-vocabulary terms, misspelled words, and rare medical terms provides a significant advantage over other models.

In embodiments, the similarity score calculator 146 calculates a similarity between vector embeddings for entities of the model domain and vector embeddings for entities of the comparison domains. Similarity matching or similarity retrieval can be used to find items, e.g., model domain entities, that are similar to a given query item, e.g., comparison domain entity. Similarity matching measures the similarity between an entity of a comparison domain and an entity of the model domain, based on certain features or characteristics, i.e., attributes, and then ranks the entity pairs by their similarity. To measure similarity, a distance measure or similarity metric is chosen. Common distance measures include Euclidean distance, cosine similarity, and Jaccard similarity. When dealing with large data sets, an index may be created to speed up the search process. An index is a data structure that organizes the data in a way that allows for efficient retrieval of similar items.

In one or more embodiments, the similarity score calculator 146 includes the Facebook AI Similarity Search (FAISS). FAISS is an open-source library developed by Facebook for efficient similarity search and clustering of high-dimensional vectors. FAISS is optimized for both CPU and GPU architectures, enabling fast and scalable similarity search operations on large datasets. FAISS supports a range of similarity metrics, including Euclidean distance, cosine similarity, inner product, and L2 distance. FAISS offers various indexing methods, including the flat index, inverted file (IVF), Hierarchical Navigable Small World (HNSW), and product quantization. Flat index uses an index built from data points without any hierarchical structure. When a search operation is performed, the distance between the query vector and all the other vectors utilized to build the index is computed and the top-n closest vectors are returned. When using IVF, a dataset is divided into clusters using a clustering algorithm (e.g., k-means). Each cluster is associated with a unique identifier. For each cluster, an inverted list is created. An inverted list is a data structure that associates a cluster identifier with the list of vectors that belong to that cluster. During indexing, each data vector is assigned to the nearest cluster centroid. This assignment is used to determine the inverted list to update with the vector's information. When performing a similarity search, the query vector is quantized to the nearest cluster centroid. FAISS then searches the inverted list associated with that cluster for potential nearest neighbors. HNSW is an algorithm for efficient similarity search in high-dimensional spaces. These indexing techniques help speed up nearest-neighbor searches in high-dimensional spaces.

In an embodiment, FAISS is combined with HNSW as the indexing approach. FAISS can be integrated with popular machine learning libraries and frameworks, such as PyTorch and TensorFlow, making it easier to incorporate similarity searches into machine learning pipelines. This may lead to significant improvements in the speed and scalability of the similarity search operations. As an open-source library, FAISS is available for developers and researchers to use, modify, and contribute to the development FAISS.

In one or more embodiments, the grouping engine 148 arranges relevant or synonymous standard medication codes into groups. The grouping engine 148 may base the groupings on TTY and/or status. For example, semantic clinic drugs are grouped with semantic branded drugs. Similarly generic packs are grouped with brand name packs. With respect to status, remapped codes may be grouped with active codes. Similarly, quantified codes may be grouped with active codes.

In one or more embodiments,. the selection engine 150 presents candidate standard medication codes for mapping to target medication free text to the user interface 106 based on the similarity values 128 provided by the similarity score calculator 146. The selection engine 150 may present an “N” number of candidate standard medication codes for mapping to a target medication free text. The “N” number of candidate standard medications codes may be ranked by the similarity values between the vector embeddings of the candidate standard medication codes and the vector embedding of the medication free text. Alternatively, the selection engine 150 presents every standard medication codes having a similarity measure above a threshold.

In one or more embodiments, the deduplication engine 152 is a software component or system designed to identify and remove duplicate medication entries from a patient's medication list or medication history. Duplicate medication entries can occur due to various factors, such as discrepancies in data entry, multiple prescriptions for the same medication, or changes in medication regimens over time. Deduplication helps ensure the accuracy, consistency, and completeness of the medication record, supporting safe and effective medication management.

The deduplication engine 152 analyzes the medication list to identify duplicate entries based on predefined criteria or rules. Common criteria for duplicate detection include medication name, dosage, strength, route of administration, frequency, and start/end dates. The deduplication engine 152 compares medication entries against each other to identify potential duplicates or near-duplicates.

In one or more embodiments, the deduplication engine 152 employs matching algorithms and similarity analysis techniques to compare medication entries and determine their similarity or equivalence. Once duplicate medication entries are identified, the engine applies deduplication strategies to resolve duplicates and consolidate redundant information. This may involve merging duplicate entries, updating existing entries with the most current information, or flagging duplicates for manual review by healthcare providers.

In embodiments, the deduplication engine 152 prioritizes duplicate medication entries based on severity, clinical relevance, or potential patient harm. The deduplication engine 152 provides decision support tools and recommendations to assist healthcare providers in resolving duplicates and making informed decisions about medication management. This may include alerts, notifications, or suggestions for medication reconciliation and rationalization. The deduplication engine 152 integrates with clinical systems, medication management tools, and EHR systems to facilitate seamless deduplication workflows. The deduplication engine 152 may present deduplicated medication lists to healthcare providers within their existing workflows, allowing users to review, validate, and finalize medication reconciliations efficiently.

In one or more embodiments, user interface 106 refers to hardware and/or software configured to facilitate communications between a user and the synchronization engine 104. User interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language, such as JavaScript. The content of user interface elements is specified in a markup language, such as hypertext markup language (HTML) or XML User Interface Language (XUL). The layout of user interface elements is specified in a style sheet language, such as Cascading Style Sheets (CSS). Alternatively, user interface 106 is specified in one or more other languages, such as Java, C, or C++.

In one or more embodiments, external sources 108 for patient records include health information exchange (HIE) 154 and healthcare databases 156.

In one or more embodiments, HIE 154 refers to the secure electronic sharing of health-related information between healthcare organizations, healthcare professionals, patients, and other authorized parties. HIE 154 enables the seamless exchange of patient health information, including medical records, test results, medication histories, treatment plans, and other clinical data, across different healthcare settings and systems. The primary goal of HIE 154 is to improve care coordination, enhance patient outcomes, and optimize healthcare delivery by ensuring that relevant and timely information is accessible to healthcare providers at the point of care. HIE systems adhere to interoperability standards and specifications that define how health information is structured, transmitted, and accessed. Common standards include HL7 (Health Level Seven), FHIR (Fast Healthcare Interoperability Resources), CCD (Continuity of Care Document), and DICOM (Digital Imaging and Communications in Medicine). HIE networks consist of participating healthcare organizations, providers, laboratories, pharmacies, payers, and other entities that contribute and exchange health information within the network. Participants may include hospitals, clinics, physician practices, long-term care facilities, public health agencies, and insurers.

In one or more embodiments, HIE systems employ secure data exchange infrastructure, such as health information exchange platforms, networks, and interfaces, to facilitate the transmission of health information between disparate systems and organizations. This infrastructure may include electronic interfaces, secure messaging protocols, health information service providers (HISPs), and data repositories. HIE systems incorporate mechanisms for obtaining patient consent and authorization for the sharing of their health information. Patients have the right to control who can access their health information and can specify their preferences regarding data sharing and disclosure. HIE initiatives implement robust data governance and security measures to protect the privacy, confidentiality, and integrity of patient health information. This includes encryption, access controls, authentication mechanisms, audit trails, and compliance with regulatory requirements such as HIPAA (Health Insurance Portability and Accountability Act). HIE systems support query-based exchange, allowing authorized healthcare providers to access patient health information from other participating organizations as needed. Providers can query the HIE network to retrieve relevant clinical data, such as medical history, allergies, medications, and lab results, to support clinical decision-making and care coordination. HIE systems facilitate directed exchange of health information between specific providers or organizations involved in the care of a shared patient. This enables targeted sharing of information, such as referrals, care transitions, and care coordination activities, while maintaining patient privacy and confidentiality.

In one or more embodiments, healthcare databases 156 refer to repositories of electronic health information that store, organize, and manage a wide range of healthcare-related data. These databases play a crucial role in various healthcare functions, including patient care, clinical research, population health management, and healthcare administration.

In one or more embodiments, the healthcare databases 156 include EHR databases, clinical data warehouses, and research databases. EHR databases store comprehensive electronic health records for individual patients, including demographic information, medical history, medications, allergies, lab results, imaging studies, clinical notes, and treatment plans. EHRs support clinical documentation, care coordination, and patient management across different healthcare settings. Clinical data warehouses aggregate and integrate data from multiple sources, such as EHRs, laboratory systems, radiology systems, pharmacy systems, and billing systems, into a centralized repository. These databases enable healthcare organizations to perform analytics, reporting, and research on large datasets to support clinical decision-making, quality improvement, and population health management. Research databases store clinical research data, including patient enrollment information, study protocols, clinical trial data, and research findings. These databases support clinical research initiatives, observational studies, comparative effectiveness research, and post-market surveillance of medical products.

In one or more embodiments, the healthcare databases 156 include registries, administrative databases, and pharmacy databases. Registries are specialized databases that collect and maintain data on specific populations, diseases, conditions, or procedures. Examples include cancer registries, diabetes registries, transplant registries, and immunization registries. Registries facilitate disease surveillance, epidemiological research, and outcomes monitoring to improve healthcare delivery and public health. Administrative databases contain administrative and billing data generated by healthcare organizations, insurers, and government agencies. These databases capture information such as patient demographics, insurance coverage, healthcare services rendered, reimbursement claims, and healthcare costs. Administrative databases support healthcare financing, reimbursement, policy analysis, and resource allocation. Pharmacy databases store data related to medication dispensing, prescription orders, medication administration, and medication usage patterns. These databases provide insights into medication adherence, prescribing trends, drug utilization, and medication safety to support medication management and quality improvement initiatives.

### 3. Presenting Recommendations of Candidate Standard Medication Codes for Mapping to Unmapped Medication Codes to Facilitate Medication Event Deduplication

FIGS. 2A-2C illustrate an example set of operations for recommending standard medication codes for mapping to unmapped medication codes to facilitate medication event deduplication in accordance with one or more embodiments. One or more operations illustrated in FIGS. 2A-2C may be modified, rearranged, or omitted all together. Accordingly, the particular sequence of operations illustrated in FIGS. 2A-2C should not be construed as limiting the scope of one or more embodiments.

One or more embodiments access patient medication data from healthcare databases for the patient, the patient medication data including medication associated with patient medication events (Operation 202). When the system detects a trigger for synchronizing a patient medication record, e.g., scheduling of an appointment or admittance to a hospital, the system queries the relevant healthcare databases to retrieve the patient's medication data. This may involve querying electronic health record (EHR) systems, pharmacy databases, medication management systems, or other clinical databases that store medication-related information. The queried data is extracted from the healthcare databases, focusing specifically on medication-related information. Medication-related information includes details such as medication names, dosage, frequency, route of administration, start and end dates, prescribing providers, dispensing pharmacies, and medication events (e.g., prescriptions, refills, administrations).

One or more embodiments crossmap the medication codes associated with the medication events with standard medication codes (Operation 204). Cross-mapping medication codes associated with medication events to standard medication codes involves translating or converting the medication codes used in a specific system or database to standardized medication codes recognized across healthcare settings. Apply the mapping algorithm or rules to cross-map the medication codes associated with medication events to standard medication codes. This process involves matching each source code to the most appropriate standard code or codes based on the defined mappings and criteria.

One or more embodiments determine if any of the unmapped medication events include free text (Operation 206). To determine if any of the unmapped medication events include free text, the system inspects the medication event data and identifies instances where the medication information is not mapped to standard medication codes and instead appears as unstructured or free-text data.

One or more embodiments remove unmapped medication events not associated with medication free text from patient medication data (Operation 208). When an unmapped medication event is not associated with a standard medication code and does not include medication free text, the unmapped medication event may be removed from the patient medication data. Alternatively, the unmapped medication events may be annotated and presented to a healthcare provider as unmapped or unknown medication events.

One or more embodiments generate a plurality of vectors embeddings for the unmapped medication codes using the free text associated with the corresponding unmapped medication codes (Operation 210). The system uses vector embedding techniques, e.g., natural language processing (NLP), to represent the text data as numerical vectors in a high-dimensional space. The text may be preprocessed to clean and standardize the text data. This may involve steps such as tokenization, lowercasing, removing punctuation, stop word removal. Pre-trained word embeddings models, e.g. SABERT, are used to represent words in the text data as dense numerical vectors. These word embeddings capture semantic relationships between words based on their context and distributional patterns in large text corpora. Individual word embeddings may be combined to generate sentence-level embeddings for the medication free text associated with each unmapped medication code. Various techniques can be used for this purpose, including averaging word embeddings, weighted averaging based on term frequency-inverse document frequency (TF-IDF). Each unmapped medication code is represented as a numerical vector or vector embedding in the high-dimensional space, capturing the semantic meaning of the corresponding free text. These vector embeddings will be used for various downstream tasks such as similarity comparison, clustering, classification, or information retrieval.

One or more embodiments access a plurality of standard medication event codes, each standard medication event code corresponding to a set of attributes (Operation 212). The standard medication event codes may be accessed through online resources, licensing the data from a vendor or organization, or utilizing open-access datasets provided by standardization bodies or government agencies. Using tools, APIs, or databases provided by the standardized medication coding system to query or browse the available medication codes. For each standard medication event code, the system retrieves the corresponding attributes associated with the code. This structured data may include information such as drug names, ingredients, strengths, dosage forms, routes of administration, indications, contraindications, adverse effects, and other relevant clinical attributes.

One or more embodiments generate a plurality of vector embeddings for the plurality of standard medication codes using text of the attributes corresponding to each standard medication event code (Operation 214). Generating the plurality of vector embeddings for the plurality of standard medication event codes includes applying the same vector embedding technique as applied to the medication free text to the text of the attributes associated with the respective standard medication event codes. The text may be preprocessed in the same manner as the text of the attributes associated with the respective standard medication codes. Individual word embeddings may be combined to generate sentence-level embeddings for the text of the attributes associated with each standard medication code.

One or more embodiments compute a similarity metric for a first target vector embedding and a first vector embedding of a first standard medication code of the plurality of standard medication event codes (Operation 216). To compute the similarity metric between the target vector embedding and the vector embedding of a standard medication code the system uses a similarity measure, e.g., cosine similarity, Euclidean distance.

One or more embodiments determine if the similarity metric for the first target vector embedding and the first vector embedding meet a threshold (Operation 218). The system determines if the similarity metric for the first target vector embedding and the first vector embedding meet the threshold. The threshold depends on the similarity measure used and the level of similarity consider significant. For cosine similarity, the value ranges from −1 to 1, where a value closer to 1 indicates higher similarity. In embodiments, a similarity metric exceeding 0.9 meets the threshold.

One or more embodiments present the first standard medication code as a candidate standard medication code for mapping to the medication free text (Operation 220). When the similarity metric of the first target vector embedding and the first vector embedding meet or exceed the threshold, the system presents the first standard medication code for mapping to the first unmapped medication code.

One or more embodiments refrain from presenting the first standard medication code as a candidate for mapping to the first unmapped medication code (Operation 222). When the similarity metric of the first target vector embedding and the first vector embedding fail to meet the threshold, the system refrains from presenting the first standard medication code for mapping to the first unmapped medication code.

One or more embodiments create a first grouping comprising the first standard medication code with one or more similar standard medication codes (Operation 224). The system identifies one or more standard medication codes that are relevant or synonymous to the first standard medication code. When RxNorm codes are used as the standard medication codes, the grouping may be according to TTY or status. For example, SCD may be grouped with SBD, and GPCK may be grouped with BPCK. Similarly, remapped codes may be grouped with active codes, and quantified codes may be mapped with active codes.

One or more embodiments present the first grouping to a user as a candidate grouping for mapping to the first unmapped medication code (Operation 226). The groupings are then presented to the user as candidates for the first unmapped medication code. The groupings may be presented in a user interface that includes the medication free text associated with the first unmapped medication code, the candidate standard medication code, the name associated with the candidate standard medication code, and the similarity metric or score. The grouping presented in the user interface may also include one or more standard medication codes similar to the candidate standard medication code, and the names associated with the one or more standard medication codes similar to the candidate standard medication code.

One or more embodiments receive user input confirming the standard medication code associated with the first grouping for mapping to the first unmapped medication code (Operation 228). The user may select the standard medication code from the user interface for mapping to the first unmapped medication code by selecting a medication code from the list, entering the code manually, or providing any additional comments or feedback. Alternatively, the system may automatically confirm a mapping of a standard medication code with a medication free text when the similarity metric is above a threshold.

In one or more embodiments, the user input confirming selection of a standard medication code for mapping to the medication free text is saved as a potential mapping for review by a terminologist. After the mapping is confirmed by the terminologist, the mapping may be added to the database of medication free text mapped to standard medication codes. In this manner, the previously unmapped medication code associated with medication free text becomes a mapped medication code as the medication free text is mapped to a standard medication code.

One or more embodiments deduplicate patient medication events associated with the mapped medication codes (Operation 230). The system uses the selected standard medication code to arrange similar or like patient medication events into buckets. Duplicate patient medication events, e.g., patient medication events having the same standard medication code as other patient medication events, may be removed. Alternatively, the duplicate patient medication events may be highlighted or otherwise annotated to indicate a potential that the patient medication event is a duplicate.

One or more embodiments present deduplicated patient medication events to a healthcare provider (Operation 232). The patient medication events may be presented to the healthcare provider in a longitudinal record or other visual form. The longitudinal record may include a first column of verified patient medication events and a second column of potential duplicates. The healthcare provider may provide indication confirming the mapping of standard medication codes to medication free text. The healthcare provider may also have the ability to provide feedback as to why confirmation was not provided for the mapping.

### 4. Example Synchronization Operation

A detailed example is described below for purposes of clarity. Components and/or operations described below should be understood as one specific example which may not be applicable to certain embodiments. Accordingly, components and/or operations described below should not be construed as limiting the scope of any of the claims.

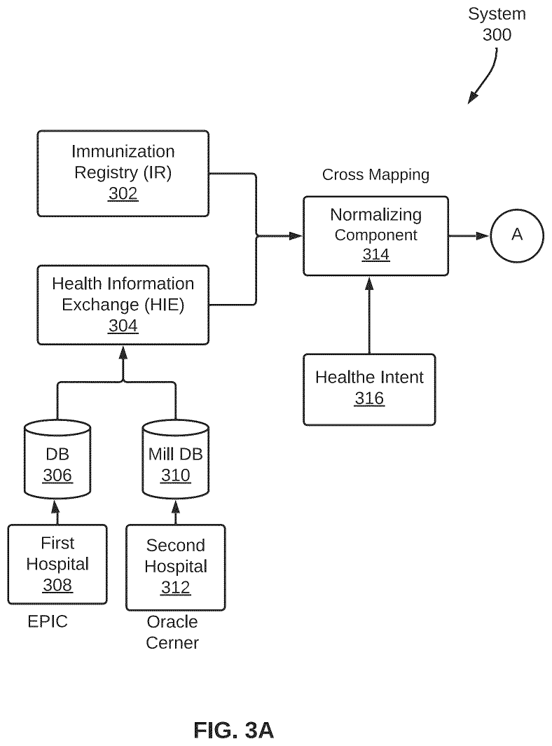

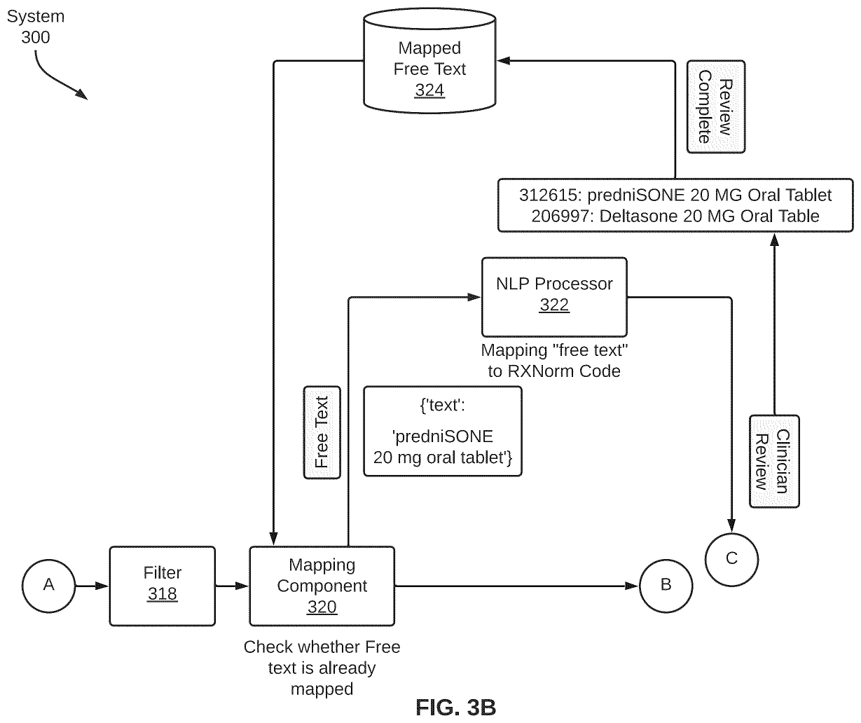

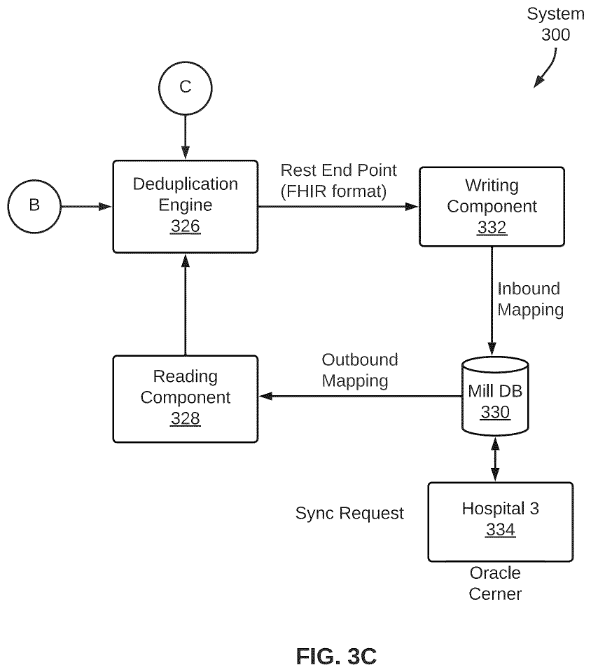

FIG. 3A illustrates a record retrieval portion of a system 300 for synchronizing a patient medication record. The record retrieval portion of system 300 retrieves patient medication information from patient records stored in the immunization registry 302 and/or from an HIE 304. As shown, the HIE 304 receives patient medication information from a database 306 associated with a first hospital 308. The first hospital 308 uses a first EHR system, e.g., EPIC. The HIE 304 may also receive patient medication information from a Millennium Database 310 associated with a second hospital 312. The second hospital 312 uses a second EHR system, e.g., Oracle Cerner.

Patient information is provided to a normalizing component 314. The normalizing component 314 may also receive patient data from Healthe Intent 316. Healthe Intent 316 is a population health management platform developed by Cerner to assist healthcare organizations manage the health of a large group of people, like a community or an entire patient base. The normalizing component 314 identifies patient medication events, crossmaps patient medication events provided as proprietary medication codes to standard medication codes.

FIG. 3B illustrates a mapping portion of the system 300. The mapping portion of the system 300 includes a filter 318 for filtering the patient medication data received from the normalizing component 314. Alternatively, filter 318 is part of normalizing component 314. The filter 318 identifies medication codes including medication free text. A mapping component 320 determines if the medication free text has already been mapped to a standard medication code.

When the medication free text has not already been mapped, the medication free text is provided to an NLP processor 322. The NLP processor 322 includes various components for preprocessing the text and generating one or more vector embeddings for the medication free text. The NLP processor 322 may also generate vector embeddings for standard medication codes and determine similarity metrics for the vector embeddings for the medication free text and the vector embeddings for the standard medication codes. The NLP processor 322 may also include a grouping component for grouping similar standard medication codes with the recommended standard medication codes. A selection component of the NLP processor 322 allows for selection of a recommended standard medication code by a healthcare provider.

A terminologist reviews the selection of the standard medication code for mapping to the medication free text. Upon determination by the terminologist that the standard medication code selected for mapping to the medication free text is accurate, the mapping of the standard medication code to the medication free text is stored in a database of mapped medication free text 324. The mapped medication free text 324 may be used by the mapping component 320 during future mapping operations of medication free text.

FIG. 3C illustrates a deduplication portion of system 300 for synchronizing patient medication data. The mapped patient medication events that were originally associated with a standard medication code or that were mapped to a standard medication code are received by a deduplication engine 326, as indicated at “B”. The deduplication engine 326 also receives the standard medication codes selected for association with the medication codes that included medication free text, as indicated at “C”. The deduplication engine 326 may also receive standard medication codes from a reading component 328. The reading component 328 receives patient medication data from a Millenium DB 330 associated with a third hospital 334 that initiated the synchronization request. The third hospital 334 uses an Oracle Cerner EHR system.

The deduplication engine 326 identifies and removes duplicate patient medication events. The deduplicated patient medication events are passed to a writing component 332 that writes the patient medication events to the Mill DB 330. A healthcare provider at the third hospital 334 access the deduplicated patient medication data from the Mill DB 330.

### 5. Example Synchronization System

A detailed example is described below for purposes of clarity. Components and/or operations described below should be understood as one specific example which may not be applicable to certain embodiments. Accordingly, components and/or operations described below should not be construed as limiting the scope of any of the claims.

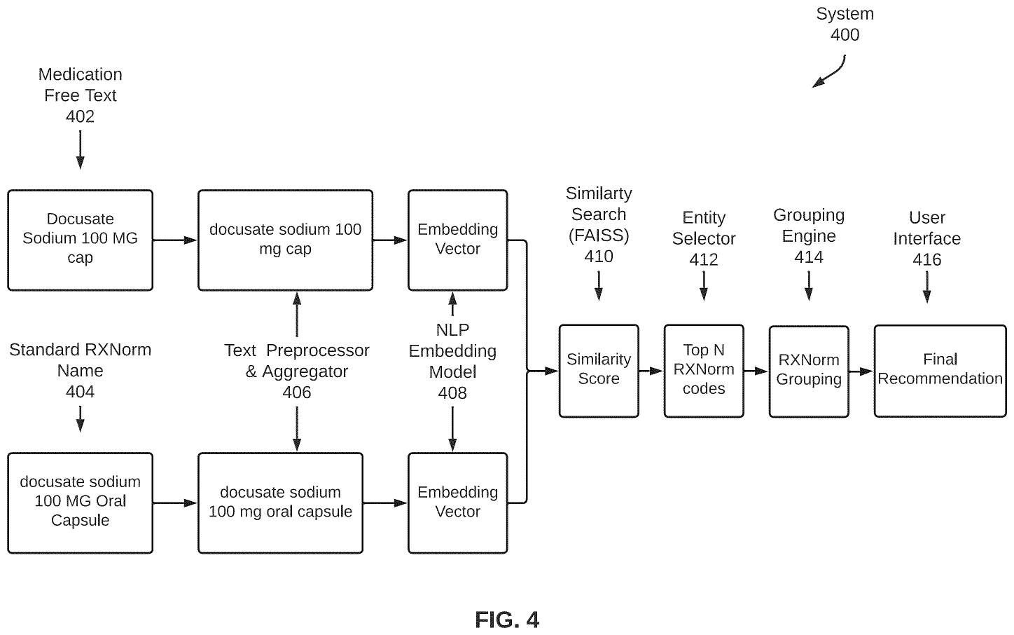

FIG. 4 illustrates operations for providing recommendations for mapping of standard medication codes to medication free text. Initially, a patient medication event associated with medication free text 402 is identified. The medication free text 402 includes “Docusate Sodium 100 MG cap”. The text for one or more attribute fields of a standard RXNorm code is identified. As shown, the standard RXNorm code attribute field is a standard RXNorm name 404. The standard RXNorm name 404 includes “docusate sodium 100 MG Oral Capsule”.

A text aggregator and preprocessor 406 is applied to the medication free text 402. Similarly, the text aggregator and preprocessor 406 is applied to the text of the standard RXNorm name 404. As shown, the text preprocessing includes lower casing the text.

An NLP embedding model 408 is applied to the preprocessed and aggregated text of the medication free text 402. The NLP embedding model 408 generates an embedding vector for the medication free text. Similarly, the NLP embedding model 408 is applied to the text of the standard RXNorm name 404 to generate an embedding vector for the standard RXNorm code associated with the standard RXNorm name 404.

A similarity search 410 calculates a similarity score between the embedding vector of the medication free text and the embedding vector of the standard RXNorm code associated with the standard RXNorm name 404.

An entity selector 412 presents the top “N” RXNorm codes for mapping to the medication free text. The top “N” RXNorm codes are ranked based on the similarity scores computed by the similarity search 410.

A grouping engine 414 provides one or more additional RXNorm codes for grouping with the recommended RXNorm codes. The additional RXNorm codes are synonymous or relevant scope to the recommend RXNorm code.

A user interface 416 displays the final recommendations of RXNorm codes for mapping to medication free text. The final recommendations of RXNorm codes may include a table listing the RXNorm name, the similarity score, and the one or more additional RXNorm codes.

### 6. Recommendation Interface

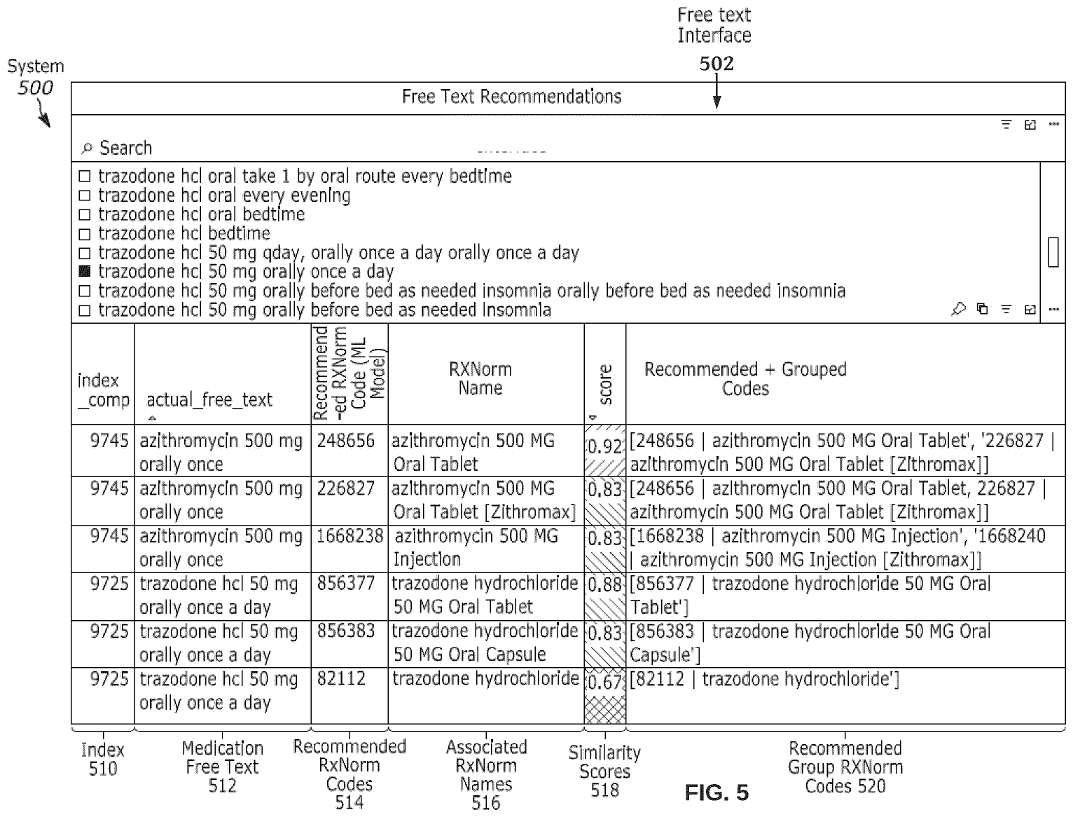

FIG. 5 illustrates an example of a recommendation interface 500 in accordance with one or more embodiments. The recommendation interface 500 may display information in a table format for easy viewing.

The recommendation interface 500 provides recommendations of RXNorm codes for medication free text. The recommendation interface 500 includes a search bar for entering a target medication free text and one or more suggested medication free text similar to the target medication free text. The one or more suggested medication free text may have a check box or other method for selecting a particular suggested medication free text.

The recommendation interface 500 also provides a table including an index 510, medication free text 512, recommended RXNorm codes 514, associated RXNorm names 516 similarity scores 518, and recommended group RXNorm codes 520. The index 510 associated with the medication free text 512. The medication free text 512 is the text entered by a healthcare provider in place of a medication code. The recommended RXNorm codes 514 are the RXNorm codes having the highest similarity scores 518. The associated RXNorm names 516 are the names for the respective recommended RXNorm codes 514.

The similarity scores 518 are the similarity values for the medication free text and the respective RXNorm codes. The recommended RXNorm codes 514 are presented in order of most similar at the top to least similar at the bottom. As shown, similarity scores above 0.90 are identified with a first color, the similarity scores between 0.90 and 0.70 are identified by a second color, and the similarity scores lower than 0.70 are identified by a third color. The recommended group RXNorm codes 520 are RXNorm codes and names that are synonymous or relevant to the respective recommended RXNorm codes 514.

### 7. Practical Application; Improvements & Advantages

Deduplication in healthcare data serves several important purposes, contributing to the overall quality, integrity, and efficiency of healthcare information systems. Deduplication ensures that each patient has a unique and accurate record, reducing the risk of administering incorrect treatments or medications. Deduplication processes help maintain the accuracy and integrity of patient information, supporting better decision-making by healthcare providers. Healthcare providers are able to access a complete and consolidated view of a patient's medical history, medications, and treatments without the interference of redundant or conflicting information. By implementing deduplication processes, healthcare organizations can provide physicians with a more reliable and consistent patient history. This, in turn, may reduce the burden on healthcare providers related to correcting and reconciling conflicting information, allowing the healthcare providers to focus more on patient care.

Duplicate records can lead to billing errors and financial discrepancies. Deduplication in healthcare data helps ensure that billing information is associated with the correct patient, preventing issues such as overbilling or underbilling. Deduplication contributes to the reliability of healthcare analytics, enabling healthcare organizations to derive insights from clean and consolidated data. Deduplicating healthcare data helps optimize resource utilization, reducing storage requirements and improving the overall performance of information systems. Deduplication supports interoperability between different healthcare systems. When patient records are consistently unique and accurate, exchanging information seamlessly between various healthcare providers, systems, and organizations becomes easier. Improved interoperability can help reduce the frustration that physicians may experience when dealing with disparate and incompatible data sources. Having a more integrated and seamless exchange of information can contribute to a smoother and less stressful workflow. Patients benefit from deduplication as it ensures that their medical information is accurate and up-to-date. This, in turn, contributes to a better patient experience, as healthcare providers can offer more personalized and effective care.

### 8. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

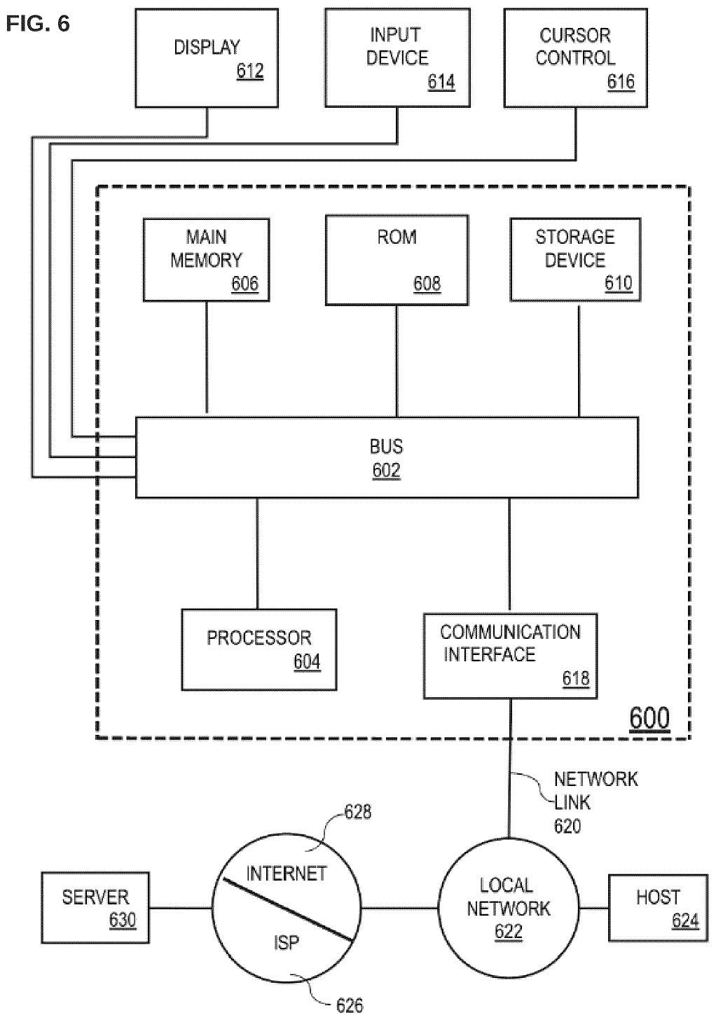

For example, FIG. 6 is a block diagram that illustrates a computer system 600 upon which an embodiment of the disclosure may be implemented. Computer system 600 includes a bus 602 or other communication mechanism for communicating information, and a hardware processor 604 coupled with bus 602 for processing information. Hardware processor 604 may be, for example, a general purpose microprocessor.

Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read only memory (ROM) 608 or other static storage device coupled to bus 602 for storing static information and instructions for processor 604. A storage device 610, such as a magnetic disk, optical disk, or a Solid State Drive (SSD) is provided and coupled to bus 602 for storing information and instructions.

Computer system 600 may be coupled via bus 602 to a display 612, such as a cathode ray tube (CRT), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane.

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 9. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a plurality of standard medication codes, each standard medication code being mapped to a corresponding set of attributes;
generating a plurality of vector embeddings corresponding respectively to the plurality of standard medication codes, wherein generating the plurality of vector embeddings comprises:
applying a machine learning model to text of a first set of attributes associated with a first standard medication code of the plurality of standard medication codes for a first medication event, to generate a first vector embedding;
accessing patient medication data of a patient from one or more sources, wherein the patient medication data comprises a target unmapped medication code corresponding to a target medication event, the target unmapped medication code comprises medication free text;
applying a machine learning model to the medication free text of the target unmapped medication code to generate a target vector embedding for the target unmapped medication code;
computing a similarity measure for the target vector embedding and each of the plurality of vector embeddings to generate a plurality of similarity measures, the plurality of similarity measures comprise:
a first similarity measure for the target vector embedding and the first vector embedding; and
based at least on the first similarity measure, presenting the first standard medication code as a candidate standard medication code for mapping to the target unmapped medication code.

2. The one or more non-transitory computer readable media of claim 1, wherein generating the plurality of vector embeddings further comprises:
applying the machine learning model to text of a second set of attributes mapped to a second standard medication code, of the plurality of standard medication codes, for a second medication event, to generate a second vector embedding;
wherein the plurality of similarity measures further comprise:
a second similarity measure for the target vector embedding and the second vector embedding;
wherein the operations further comprise:
based at least on the second similarity measure, refraining from presenting the second standard medication code as a candidate standard medication code for mapping to the target unmapped medication code.

3. The one or more non-transitory computer readable media of claim 1, wherein the patient medication data further comprises a second medication event, wherein a second standard medication code corresponds to the second medication event, wherein the operations further comprise:
identifying that the second standard medication code associated with the second medication event is the same as the first medication event associated with the first standard medication code; and
removing one of the first or second medication event from the patient medication data as duplicative of the other of the first or second medication event.

4. The one or more non-transitory computer readable media of claim 1, the operations further comprising,
identifying one or more standard medication codes that are similar to the first standard medication code;
generating a first grouping comprising the first standard medication code and the one or more similar standard medication codes,
wherein presenting the first standard medication code further comprises presenting the first grouping.

5. The one or more non-transitory computer readable media of claim 4, wherein the similarity comprises a name brand medication for a generic medication or a generic medication for a name brand medication.

6. The one or more non-transitory computer readable media of claim 1, wherein the first similarity measure comprises a weighted cosine similarity measure for the target vector embedding and the first vector embedding.

7. The one or more non-transitory computer readable media of claim 1, wherein the operations further comprise:
identifying n highest similarity measures of the plurality of similarity measures; and
presenting standard medication codes, mapped to embedding vectors that correspond to the n highest similarity measures, as candidate standard medication codes for mapping to the target unmapped medication code.

8. The one or more non-transitory computer readable media of claim 1, wherein the operations further comprise:
identifying a subset of similarity measures, of the plurality of similarity measures, that meet a threshold similarity measure; and
presenting standard medication codes, mapped to embedding vectors that correspond to the subset of similarity measures, as candidate standard medication codes for mapping to the target unmapped medication code.

9. The one or more non-transitory computer readable media of claim 1, applying the machine learning model to a target medication event comprises using one or more of the following word embedding techniques: BioWordVec fastText or Self-Alignment Pretraining for Biomedical Entity Representations (SAPBERT).

10. A method comprising:
accessing a plurality of standard medication codes, each standard medication code being mapped to a corresponding set of attributes;
generating a plurality of vector embeddings corresponding respectively to the plurality of standard medication codes, wherein generating the plurality of vector embeddings comprises:
applying a machine learning model to text of a first set of attributes associated with a first standard medication code of the plurality of standard medication codes for a first medication event, to generate a first vector embedding;
accessing patient medication data of a patient from one or more sources, wherein the patient medication data comprises a target unmapped medication code corresponding to a target medication event, the target unmapped medication code comprises medication free text;
applying a machine learning model to the medication free text of the target unmapped medication code to generate a target vector embedding for the target unmapped medication code;
computing a similarity measure for the target vector embedding and each of the plurality of vector embeddings to generate a plurality of similarity measures, the plurality of similarity measures comprise:
a first similarity measure for the target vector embedding and the first vector embedding; and
based at least on the first similarity measure, presenting the first standard medication code as a candidate standard medication code for mapping to the target unmapped medication code,
wherein the method is performed by at least one device including a hardware processor.

11. The method of claim 10, wherein generating the plurality of vector embeddings further comprises:
applying the machine learning model to text of a second set of attributes mapped to a second standard medication code, of the plurality of standard medication codes, for a second medication event, to generate a second vector embedding;
wherein the plurality of similarity measures further comprise:
a second similarity measure for the target vector embedding and the second vector embedding; and
based at least on the second similarity measure, refraining from presenting the second standard medication code as a candidate standard medication code for mapping to the target unmapped medication code.

12. The method of claim 10, wherein the patient medication data further comprises a second medication event, wherein a second standard medication code corresponds to the second medication event, further comprising:
identifying that the second standard medication code associated with the second medication event is the same as the first medication event associated with the first standard medication code; and
removing one of the first or second medication event from the patient medication data as duplicative of the other of the first or second medication event.

13. The method of claim 10, further comprising,
identifying one or more standard medication codes that are similar to the first standard medication code;
generating a first grouping comprising the first standard medication code and the one or more similar standard medication codes,
wherein presenting the first standard medication code further comprises presenting the first grouping.

14. The method of claim 13, wherein the similarity comprises a name brand medication for a generic medication or a generic medication for a name brand medication.

15. The method of claim 10, wherein the first similarity measure comprises a weighted cosine similarity measure for the target vector embedding and the first vector embedding.

16. The method of claim 10, further comprising:
identifying n highest similarity measures of the plurality of similarity measures; and
presenting standard medication codes, mapped to embedding vectors that correspond to the n highest similarity measures, as candidate standard medication codes for mapping to the target unmapped medication code.

17. The method of claim 10, further comprising:
identifying a subset of similarity measures, of the plurality of similarity measures, that meet a threshold similarity measure; and
presenting standard medication codes, mapped to embedding vectors that correspond to the subset of similarity measures, as candidate standard medication codes for mapping to the target unmapped medication code.

18. The method of claim 10, applying the machine learning model to a target medication event comprises using one or more of the following word embedding techniques: BioWordVec fastText or Self-Alignment Pretraining for Biomedical Entity Representations (SAPBERT).

19. A system comprising:
at least one device including a hardware processor;
the system being configured to perform operations comprising:
accessing a plurality of standard medication codes, each standard medication code being mapped to a corresponding set of attributes;
generating a plurality of vector embeddings corresponding respectively to the plurality of standard medication codes, wherein generating the plurality of vector embeddings comprises:
applying a machine learning model to text of a first set of attributes associated with a first standard medication code of the plurality of standard medication codes for a first medication event, to generate a first vector embedding;
accessing patient medication data of a patient from one or more sources, wherein the patient medication data comprises a target unmapped medication code corresponding to a target medication event, the target unmapped medication code comprises medication free text;
applying a machine learning model to the medication free text of the target unmapped medication code to generate a target vector embedding for the target unmapped medication code;
computing a similarity measure for the target vector embedding and each of the plurality of vector embeddings to generate a plurality of similarity measures, the plurality of similarity measures comprise:
a first similarity measure for the target vector embedding and the first vector embedding; and
based at least on the first similarity measure, presenting the first standard medication code as a candidate standard medication code for mapping to the target unmapped medication code.

20. The system of claim 19, wherein generating the plurality of vector embeddings further comprises:
applying the machine learning model to text of a second set of attributes mapped to a second standard medication code, of the plurality of standard medication codes, for a second medication event, to generate a second vector embedding;
wherein the plurality of similarity measures further comprise:
a second similarity measure for the target vector embedding and the second vector embedding;
wherein the operations further comprise:
based at least on the second similarity measure, refraining from presenting the second standard medication code as a candidate standard medication code for mapping to the target unmapped medication code.
